(19) United States
(12) Patent Application Publication
(10) Pub. No.: US 2021/0138449 A1
(43) Pub. Date: May 13, 2021

(54) OZONE CABINET

(71) Applicant: Pure Quest Technologies, Las Vegas, NV (US)

(72) Inventor: Bruce Hinkle, Newport Beach, CA (US)

(21) Appl. No.: 16/855,688

(22) Filed: Apr. 22, 2020

Related U.S. Application Data
(60) Provisional application No. 62/933,182, filed on Nov. 8, 2019.

(51) Int. Cl. B01L 1/02; A61L 2/20; A61L 2/24; F24F 7/06

(52) U.S. Cl. CPC B01L 1/025; A61L 2/202; A61L 2/24; F24F 7/06; B01L 2300/048; A61L 2202/122; A61L 2202/14; B01L 2200/0684; B01L 2200/0689; A61L 2202/121

(57) ABSTRACT

A decontamination appliance includes a control cabinet having a control circuit and a decontamination cabinet. The appliance also includes an ozone generator that supplies ozone to the decontamination cabinet. The ozone generator is controlled by the control circuit. A hydrogen peroxide vaporizer is also controlled by the control circuit. A circulation fan circulates a stream of air through the decontamination cabinet. The circulation fan circulates ozone generated from the ozone generator and hydrogen peroxide paper generated from the hydrogen peroxide vaporizer. The decontamination cabinet further has includes trays mounted on a tray rack, and the appliance is mounted on wheels for portable use. The decontamination appliance also preferably includes a humidifier.

This application is a non-provisional of and claims priority to U.S. application 62/933,182 filed Nov. 8, 2019, entitled Ozone Cabinet by same inventor Bruce Hinkle, the disclosure of which is incorporated herein by reference.

### FIELD OF THE INVENTION

The present invention is in the field of ozone plant product decontamination cabinets.

### DISCUSSION OF RELATED ART

A variety of different medical and agricultural products often require product decontamination before sale to consumers. Different decontamination techniques such as advanced oxygen processing can kill microbes. Ozone has been USDA and FDA approved for use on foods and surfaces. Many hospitals and biological laboratories have used ozone for sterilizing equipment and surfaces. Although it is well-known that both ozone and hydrogen peroxide can kill pathogens, the effectiveness of each alone is limited as seen in related art patent publications.

For example, in the U.S. Pat. No. 10,212,956 Compositions And Method Of Treating Edible Matter And Substrates Therefor by inventor Nimrod Ben Yehuda, registered Feb. 26, 2019 the abstract discloses, “There are provided methods of treating edible matter comprising applying a composition comprising performic acid to the edible matter or a substrate therefor. Other embodiments are also disclosed.”

For example, in the United States publication number 2001/0043898 Method And Apparatus For Use of Reached Hydrogen Peroxide Compounds In Industrial Process Waters by inventor Michael J. Soltz et al., published Nov. 22, 2001 the abstract discloses, “Methods and apparatus are described which utilize an aqueous hydrogen peroxide composition of hydrogen peroxide and at least one additive which serves to catalyze the rapid decomposition of the hydrogen peroxide into hydroxyl radicals. When contacted with an atmospheric effluent containing odor and/or noxious components, the hydroxyl radicals formed oxidize the odor and noxious components to non-odor offensive, environmentally acceptable by-product.”

For example, in the United States publication number 2003/0075513 Catalytic Fixed Bed Reactor Systems For The Destruction Of Contaminants In Water By Hydrogen Peroxide And Ozone by inventor Christopher M. Miller, published Apr. 24, 2003 the abstract discloses, “A process for the treatment of an aqueous media having contaminants therein, including adding hydrogen peroxide to the aqueous media, exposing the aqueous media to a catalytic environment, removing the aqueous media from the catalytic environment prior to the aqueous media having a five minute residence time within the catalytic environment, thereafter adding ozone to the aqueous media, and allowing the ozone and aqueous media to interact outside of the presence of the catalytic environment.”

For example, in the United States publication number 2017/0238568 Insulated Container Including Ozone Producing Device by inventor Scott A. Elrod et al., published Aug. 24, 2017 the abstract discloses, “Systems and methods for detecting presence of a thing, the thing treated with a descenter, one method including in an area in which it is possible that a thing treated with a descenter is present, using a detector to detect a descenter level in the area, producing a detected descenter level, comparing the detected descenter level to a normal descenter level for the area, determining that the detected descenter level is different from the normal descenter level, said determining indicating the possible presence of a thing treated with a descenter; and using a trained service animal to facilitate such a method.”

For example, in the United States publication number 2017/0304476 Sterilization Device And Methods by inventor Daniel S. Taggart et al., published Oct. 26, 2017 the abstract discloses, “Embodiments of the present disclosure generally relate to devices and methods for sterilizing equipment. More particularly, one or more embodiments described in the present disclosure are directed to portable devices for sterilizing medical equipment in emergency situations. The sterilization devices and method of the present disclosure address an unmet need for sterilizing surgical equipment in a manner that is not only effective and time-efficient, but is also portable and reliable enough to use in emergency medical situations in remote locations where modern sterilization equipment is not available.”

### SUMMARY OF THE INVENTION

The present invention uses a combination of ozone and hydrogen peroxide to create radical hydroxyls to kill microbes and spores by penetrating the cellular walls while leaving no residue. The present invention uses a combination of ozone and vaporized hydrogen peroxide in tandem to produce a synergistic effect to maximize radical hydroxyl generation in a mixture of vaporized hydrogen peroxide and ozone in one easy to use appliance.

The present invention is an appliance that has various modes of operation which cycle automatically including a humidification cycle, a vaporized hydrogen peroxide cycle, and an ozone generation cycle.

The present invention has a first mode that generates and circulates low percentage vaporized hydrogen peroxide. The preferred liquid concentration is less than 8% percent hydrogen peroxide in water. The hydrogen peroxide is kept in a reservoir and vaporized using a vaporizer such as an ultrasonic vaporizer. The hydrogen peroxide reservoir emits a stream of vaporized hydrogen peroxide. The stream of vaporized hydrogen peroxide is immediately entrained in an airflow and circulated through the decontamination chamber by circulating fans. The circulating fans maintain a constant vaporized hydrogen peroxide fog in the decontamination chamber when the decontamination chamber is active. The decontamination chamber can be formed as an appliance having a cabinet with caster wheels for portable use. Preferably, the decontamination chamber is made of stainless steel, but it can also be made from acrylic panels that can be formed to shape.

When the level of the hydrogen peroxide drops, a hydrogen peroxide filling port formed on a side of the control cabinet can provide a conduit for refilling the hydrogen peroxide reservoir. The port can have a one-way valve for easy addition of hydrogen peroxide. A gauge showing the level of the hydrogen peroxide remaining is also preferably formed on the side of the control cabinet next to the hydrogen peroxide filling port.

A control circuit monitors the operation of the decontamination chamber and turns on the ozone generators after a set time such as after half an hour. Ozone's high reactivity, penetrability and spontaneous decomposition back into oxygen makes ozone a viable disinfectant for use in food production and other areas where decontamination is needed. Ozone generators have fans that circulate air through a high-energy source, such as UV light or corona discharge to constantly generate ozone. Preferably, uses ceramic corona discharge plates within the ozone generators generate a stream of ozonated air that is then circulated to the decontamination chamber by circulation fans.

The control circuit has controls and indicators. The indicators are preferably control lights that indicate a status of the control circuit operation. The indicator lights can indicate the status of the decontamination cycle.

In the present invention, a decontamination cycle can include three phases that are automatically processed in a one-step user command. In a first phase, the decontamination chamber is humidified through a vapor cycle 70-80% by the hydrogen peroxide solution. A humidity sensor in the air chamber can monitor the humidity level to confirm operation to the control circuit. A display monitor is preferably mounted on the outside of the control cabinet at a control panel.

In a second phase, the timed control of the control circuit activates the ozone generators. Ozone concentration sensors can provide an indication of ozone concentration during this phase. The second phase ozonated air concentration increases rapidly to 25 ppm (parts per million) and is maintained at an optimum biocidal level by the ozone generator and system controls at a set timed cycle. The humidity level monitors the level of vaporized hydrogen peroxide while an optional ozone concentration sensor monitors the ozone concentration.

At the end of the decontamination cycle the control circuit cycle timer activates the exhaust fan to expel all ozonated air from the cabinet through a catalytic converter compartment so that the user can open the cabinet to retrieve the agricultural product. The load chamber is a hollow portion formed in the cabinet. Preferably, the catalytic converter compartment converts the high concentration ozone air to regular air before exhausting the chamber air to the external environment. A variety of commercially available ozone catalysts can be used in the ozone catalytic converter compartment.

Cycle times vary depending on the area volume, desired level of decontamination and area contents, but are typically between 120 min and 190 min. For cannabis, a cabinet can process approximately 10-12 lbs. per two-hour cycle. Different weight of biological material can be accounted for with a 3 position selector switch that can activate different time modes. For example a first time light indicator indicates the operation of a first time mode. A second time light indicator indicates the operation of a second time mode, and a third time indicator indicates the operation of a third time mode. For example, in a first schedule, there can be an initial vaporized hydrogen peroxide phase followed by an ozone generation phase and then an exhaust phase after two hours. In a second schedule, the ozone generation phase can be four hours and in a third schedule, the ozone generation phase can be in six hours.

The keypad can be configured to modify the timing of the 3 position selector switch. For example, a code for different species, breed or variety of agricultural products can provide a multi-species preprogrammed setting that changes the 3 position selector switch to different timings such as instead of in two hour intervals, perhaps in 45 minute intervals or in one hour intervals.

Timing can also be modified according to the weight of the agricultural product in the decontamination cabinet. A digital scale optionally provides the control circuit with knowledge of the weight of the agricultural product. This digital scale can then automatically modify the selector switch setting to have the right timing. The cabinet preferably has room for the agricultural product to be decontaminated. The agricultural product can be placed on a vertical array of stainless steel trays sliding mounted on a rack so that each level of tray is removable from the rack.

The ozone generation is preferably controlled at a control panel. The control panel has a power light indicator. An on off switch visually indicates the power supply. A cycle time switch control can select between different discrete cycle times such as a first program mode, a second program mode and a third program mode. The ozone generation and control panel are preferably mounted in the control cabinet which is adjacent to the decontamination cabinet. A variety of vented openings connect the ozone generation cabinet to the control cabinet.

A system light indicator indicates when the system is activated. A safe entry light indicator can provide an indication as to when the chamber is safe for opening. The decontamination cabinet chamber has doors that have automatic locks. The automatic locks lock the door until the exhaust system activates. A vapor light indicator indicates when the vapor is on. A lock light indicator indicates the locking status of the cabinet, and an exhaust light indicator indicates the status of the exhaust fan.

An access control system that reads a preprogrammed code or wireless key, can have a 10 key keypad for entering codes and controlling access to the system. The ozone generation cabinet is made of stainless steel and has a recess for the access control system and indicator lights. Preferably, a control cabinet includes the ozone generators and the vaporized hydrogen peroxide generator.

The key feature of the present invention is the one touch activation of the process. Once the cabinet is loaded, a user need only press the start button once to start and complete the entire process. Once the process is completed an indication can be given to the user such as a completion indicator light or an audible signal such as a beep. After the first process is completed, the user reloads the cabinet and need only press the start button again. Thus, the cabinet provides a completely automatic decontamination or sterilization factory that is a portable appliance with the ease of a one touch start button. This allows untrained users to operate the decontamination appliance without error.

### BRIEF DESCRIPTION OF THE DRAWINGS

FIG. 1 is a diagram of the control cabinet interior configuration.

FIGS. 2, 3 are diagrams of the catalytic converter chamber used for exhaust in the form of a filter box.

FIG. 4 is a diagram of the control cabinet side exterior configuration.

FIG. 5 is a diagram of the control cabinet rear exterior configuration that is facing the decontamination chamber.

FIG. 6 is a wiring diagram of the control circuit.

FIG. 7 is a left front exterior view of the appliance.

FIG. 8 is a right front exterior view of the appliance.

FIG. 9 is a general diagram of the appliance control panel.

The following callout list of elements can be a useful reference for call out numbers of the drawings.

20 control cabinet
21 caster wheels
22 cabinet door
23 control panel
24 key pad
25 temperature indicator
26 left air column
27 right air column
30 decontamination chamber
31 tray
32 tray rack
33 product
34 door latch
35 warning sign
36 decontamination chamber door
37 manual latch
39 door viewing window
40 door indicator
41 main power on
42 three position selector switch
43 system on/off
44 two hour timer
45 four hour timer
46 six hour timer

### 47 GFI 15A

48 power on light
49 O3 GEN on light
50 relay 10A
51 O3 GEN fans
52 O3 GEN timer 1
53 O3 GEN timer 2
54 O3 generator
55 door latch
56 recap inlet
57 low voltage 12vo transformer
58 O3 generator
59 vapor timer
60 door control
61 door lock on
62 neutral blocks
63 vapor on light
64 vapor unit
65 exhaust on light
66 exhaust timer
67 exhaust fan
68 exhaust relay
69 system light
71 three position selector switch load line
72 first system load line
73 second system load line
74 second system load line
75 six hour timer load line
76 four hour timer load line
78 two hour timer load line
79 first relay load line
80 second relay load line
81 third relay load line
82 power on light load line
83 low-voltage transformer load line
84 ozone generator on light load line
85 ozone generator fan load line
86 exhaust timer load line
87 vapor timer load line
88 second ozone generator timer load line
89 first ozone generator timer load line
90 ozone generator load line
91 vapor unit load line
92 vapor on light load line
93 exhaust on light load line
94 door control 12 V low line
95 door lock 12 V low line
96 door latch 12 V low line
97 recapture inlet load line
98 ground fault interrupt load line
99 exhaust relay load line
100 exhaust fan load line
101 ozone generator
102 air vent filter
103 recessed control board
104 recessed reservoir fill
105 reservoir fill line
106 hydrogen peroxide reservoir
107 reservoir sight glass
108 reservoir hydrogen peroxide pump to vaporizer
109 vaporizer hydrogen peroxide generator
110 vapor outlet
111 vaporizer hydrogen peroxide level indicator
112 reservoir pump filling tube
113 exhaust axial fan
114 filter box exhaust port gasket
115 filter box
116 component mounting board
117 ozone generator mounting receiver
120 exhaust fan ground line
121 vapor unit ground line
122 ozone generators ground line
123 low-voltage 12 V transformer ground line
124 ground fault interrupt ground line
125 recapture inlet ground line
126 ground line
130 door control 12 V high line
131 door latch 12 V high line
132 door lock 12 V high line
138 thermometer
140 six hour timer neutral line
141 four hour timer neutral line
142 ozone generator fan neutral line
143 first ozone generator timer neutral line
144 second ozone generator timer neutral line
145 vapor timer neutral line
146 door latch control line
147 two hour timer neutral line
148 ground fault interrupt 15 amp neutral line
149 neutral block line
150 exhaust timer neutral line
151 exhaust fan neutral line
152 exhaust on light neutral line
153 vapor unit neutral line
154 vapor on light neutral line
155 ozone strobe light neutral line
156 ozone generator on light neutral line
157 power on light neutral line
158 low-voltage 12 V transformer neutral line
200 filter media box 18 ga stainless steel
201 media box (filled with Carulite 15 lbs ozone distribute material to ½ from the top)
202 stainless steel mesh screen
203 stainless steel baffle structure (stainless steel 18 ga)
204 ½ EDPM gasket around top
205 first baffle
206 second baffle
301 install bottom of control cabinet
302 control cabinet side panel
303 control cabinet back panel
304 exhaust outlet
305 ozone inlet
401 air vents
402 exhaust vent
403 power plug inlet
404 hydrogen peroxide filling access
405 hydrogen peroxide sight gauge indicator
406 control panel
500 control cabinet rear
501 vent hole
502 vapor outlet
503 cabinet back

### DETAILED DESCRIPTION OF THE PREFERRED EMBODIMENT

As seen in FIG. 7, the control cabinet 20 is mounted on caster wheels 21 so that it can be moved around on a flat floor. The control cabinet 20 has a cabinet door 22 for accessing internal components within the control cabinet 20. A control panel 23 is mounted to an exterior vertical face of the control cabinet 20. The control panel 23 includes indicator lights and a keypad 24. Additionally, a temperature indicator 25 can indicate temperature of thermocouples at a variety of different locations, such as the ozone generator 101, the vaporized hydrogen peroxide generator 109, the cabinet tray rack 32 or the tray 31. The control cabinet 20 has a control cabinet rear 500. The control cabinet rear 500 is connected to the decontamination chamber 30.

The decontamination chamber 30 has trays 31 mounted on tray racks 32. Product 33 to be decontaminated is held in the trays 31. Each of the trays have a number of openings within the trays for allowing airflow. A vaporized hydrogen peroxide generator 109 can be placed in the decontamination chamber 30, and a door latch 34 can provide an electronic locking mechanism. For further securing the door, a manual latch 37 can provide additional door stability.

As seen in FIG. 8 the control cabinet 20 has a cabinet back 503 that connects to a decontamination chamber. The decontamination chamber has a left air column 26 and a right air column 27 to allow air circulation around the tray rack 32 and the trays 31. Preferably, a warning sign 35 or other indicator can be placed on the door viewing window 39 of the decontamination chamber door 36 so as to provide a warning against opening the door in the middle of a cycle. The automatic door latch 34 can discourage a user from opening the door in the middle of a cycle. The door latch 34 has a door latch control cable 38 that passes to the electronic controller. The electronic controller can be implement it on a PCB.

As seen in FIG. 4, the cabinet back 500 has a layout that includes a vent hole 501 to provide a stream of ozone, and a vapor outlet 502 to provide a stream of hydrogen peroxide vapor or mist. The vent hole 501 circulates ozone from an ozone generator 101, FIG. 1 and the vapor outlet 502 of the cabinet back preferably connects to the vapor outlet 110 of the hydrogen peroxide vapor generator.

As seen in FIGS. 2, 3, a filter media box 200 facilitates ozone disposal. The ozone after usage exits the decontamination chamber from an ozone inlet 305 and exits the cabinet through an exhaust outlet 304. Preferably, the ozone inlet 305 is on the control cabinet back panel 303, and the exhaust outlet 304 is mounted on the control cabinet side panel 302. The install bottom of control cabinet 301 is the bottom side of the filter media box. The filter media box 200 also can fit within the control cabinet. A filter insert can be placed within the filter media box. The filter insert may have a first baffle 205 and a second baffle 206 that catalyzes hydrogen peroxide and ozone into more inert substances. Preferably, the filter media box 200 has a gasket 204 which is a ½ EDPM gasket around top around the top edge of the filter media box. A media box insert 201 preferably has stainless steel baffle structure 203 which can be fine-mesh of 18 gauge stainless steel. The media box insert 201 is preferably filled with carulite of approximately 15 pounds that is filled about half way to the top of the media box insert 201. The media box insert 201 can be made as a tray that is removable from the filter box 200, or can be integrally formed with the filter box 200. Preferably, a stainless steel mesh screen 202 can be mounted on the ozone intake opening.

As seen in FIG. 1, the control cabinet 20 is configured as a stainless steel shelving was various levels. A pair of ozone generators 101 can be mounted on ozone generator mounting receivers 117 that are formed as brackets for retaining the pair of ozone generators 101. The control cabinet 20 has air vent filters 102 that allow for air intake from outside of the cabinet. The air vent filters can be mounted on the left and right sides of the control cabinet 20. Additionally, a recessed control board 103 can also be mounted on the left and right sides of the control cabinet 20. The recessed control board can have electronics in it that control all of the components of the appliance. For example, the control board can control the ozone generators and the vaporizer. The side of the control cabinet 20 may also have a funnel recess to provide a recessed reservoir fill 104 that allows a user to deposit hydrogen peroxide into a hydrogen peroxide reservoir 106 from a side of the cabinet without opening the cabinet door. A reservoir site glass 107 provides a view of the height of the reservoir. The reservoir fill line 105 denotes a line to which the hydrogen peroxide should be filled up to. A reservoir hydrogen peroxide pump to vaporizer 108 pumps the hydrogen peroxide from the hydrogen peroxide reservoir 106 to the vaporizer hydrogen peroxide generator 109. The vaporizer hydrogen peroxide generator 109 can be an ultrasonic vaporizer that has a vaporizer hydrogen peroxide level indicator 111 that is preferably maintained at a constant level for optimizing the effect of the ultrasonic vaporizer element submerged in the hydrogen peroxide. After hydrogen peroxide vapor is generated at the vaporizer hydrogen peroxide generator 109, the hydrogen peroxide vapor can be introduced into the decontamination chamber at the vapor outlet 110. As hydrogen peroxide is consumed in the vaporizer 109, additional hydrogen peroxide can be refilled from the reservoir, such as by a reservoir pump filling tube 112.

The filter box 115 is preferably placed on the lower level and has a filter box exhaust port gasket 114 that makes a seal to an exhaust axial fan 113. The axial fan 113 activates when the entire procedure is finalized and the axial fan 113 draws hydrogen peroxide vapor and ozone from the interior of the cabinet.

As seen in FIG. 4, air vents 401 mounted on a side of the control cabinet 20 allow aspiration of air into the system. An exhaust vent 402 exhausts the decontamination chamber through the filter box 115. A power plug inlet 403 provides power to the cabinet. Preferably, the cabinet can be operated on household electric current of 120 V or 240 V. The side of the cabinet also has a hydrogen peroxide filling access 404 and a hydrogen peroxide sight gauge indicator 405. The control panel 406 provides a user with indicators to indicate operation as well as controls for controlling the appliance.

As seen in FIG. 9, the control panel has a variety of different indicators and controls. A keypad provides input to the system. The keypad can also have an access card that has radiofrequency identification or transponder capability for operating specific programs. The key cards can be assigned to different people and colored coded for running specific programs. Thus, a user need only enter a pin number on the keypad or swipe the card to operate the appliance. Once operational, the appliance performs all of the preset cycles like any other appliance such as a residential clothes washer or clothes dryer.

A three position selector switch 42 selects the different modes of operation such as different hours that the machine will run. The system on-off switch 42 provides a power control that is indicated at a power on light 48. A vapor indicator 63 indicates vapor generation, and an and ozone generator light 49 indicates ozone generation. A system light 69 indicates that the system is on. A two hour timer light 44 indicates the two hour cycle, a four hour timer light 45 indicates a four hour cycle, and a six hour timer light 46 indicates a six hour cycle. A door indicator light 40 indicates whether the door is open, and a doorlock on light 61 indicates whether or not the door is locked. An exhaust on light 65 indicates whether or not the exhaust is operational. A thermometer 138 may also be mounted on the control panel and have a variety of different thermocouples for taking temperature of different processes within the system.

Electrical implementation of the system is shown in FIG. 6 which is a circuit diagram of the present invention. The controls in the electrical implementation of the system can be electronically controlled such as by a microcontroller. A main power on 41 provides an AC load line for a variety of different components, such as the three position selector switch 42, the power on light 48. The main power on 41 is protected by a GFI 15 A 47 which is a ground fault interruption of 15 amperage in rating. The recap inlet 56 is the power plug that connects to outside of the machine and provides a ground to a ground block and a neutral to the neutral block 62.

A three position selector switch 42 selects between a two hour timer 44, a four hour timer 45 and a six hour timer 46. The two hour timer 44, the four hour timer 45 and the six hour timer 46 connect to the 10 amp relay 50 which controls and switches power to the ozone generation fans 51, the first ozone generation timer 52, the second ozone generation timer 53, the exhaust timer 66, and the ozone generation on light 49. The ozone generators 54 are controlled by the first ozone generation timer in the second ozone generation timer. The ozone generators 54 can be wired in parallel so that for example three ozone generation devices can operate simultaneously.

The system on-off switch 43 activates and powers a low-voltage 12 V transformer 57 which in turn powers the door control 60. The door control 60 controls the doorlock on indicator light 61 and the door latch 55. Exhaust timer 66 controls the exhaust on light 65 and the exhaust relay 68, which controls the exhaust fan 67. The vapor timer 59 controls the vapor generation unit 64 and the vapor on light 63. Neutral blocks 62 provide a wire connection means for connecting neutral lines to blocks.

The wiring between the components includes neutral lines, load lines, ground lines and direct current voltage lines. The recapture inlet load line 97 provides power to the ground fault interrupt which then provides power to the main power by the ground fault interrupt load line 98. The three position selector switch load line 71 connects between and sends an AC load from the main power to the three position selector switch. The three position selector switch selectively provides a first system load line 72 connected to the six hour timer by the six hour timer load line 75, a second system load line 73 connected to a four hour timer load line 76, or a third system load line 74 connecting to a two hour timer load line 78. In between the three position selector switch and the timers is a system on-off switch 43 that may also be an indicator such as a switch with an indicator light mounted on it. The timers are connected to the relay at a first relay load line 79, a second relay load line 80, and a third relay load line 81. When power is on, a power on light load line 82 provides a load to a power on light.

The system switch optionally provides a low-voltage transformer load line 83 which then provides DC power to a door control via a door control 12 V high line 130. The high line is preferably 12 V in contrast to a low line that is 0 V. A control line can be a voltage different than the high line or the low line. The door control has a door latch 12 V high line 131 that provides a 12V DC power to the door latch. The door control 12 V low line 94 provides a low-voltage line to the door control which can be a ground. The door latch 12 V low line 96 provides a low-voltage line to the door latch which can be a ground. The doorlock 12 V low line 95 provides a low-voltage line to the door lock. The low-voltage 12 V transformer ground line 123 provides a ground line for the low-voltage 12 V transformer. A door lock 12 V high line 132 provides a 12V DC power to a doorlock.

The ozone generator on light load line 84 provides a load line to the ozone generator on light. The ozone generator fan load line 85 provides a load line to the ozone generator fan. The exhaust timer load line 86 provides a load line to the exhaust timer. The vapor timer load line 87 provides a load line to the vapor timer. The second ozone generator timer load line 88 provides a load line to the second ozone generator timer. The first ozone generator timer load line 89 provides a load line to the first ozone generator. The ozone generator load line 90 provides a load line to the ozone generator. The vapor unit load line 91 provides a load line to the vapor unit. The vapor on light load line 92 provides a load line to the vapor on light. The exhaust on light load line 93 provides a load line to the exhaust on light. The exhaust relay load line 99 provides a load line to the exhaust relay. The exhaust fan load line 100 provides a load line to the exhaust fan.

Similarly, an exhaust fan ground line 120 provides a ground line to the exhaust fan. A vapor unit ground line 121 provides a ground line to the vapor unit. An ozone generator ground line 122 provides a ground line to the ozone generators.

A ground fault interrupt ground line 124 provides a ground line for the ground fault interrupt. A recapture inlet ground line 125 provides a ground line from the recapture inlet to the ground block. A ground line 126 provides a ground line to the recapture inlet.

Neutral lines are also provided from a pair of neutral blocks as shown in the electrical diagram. A neutral block line 149 can connect the pair of neutral blocks to each other, and the pair of neutral blocks can be electrically connected to the recap inlet. The recap inlet is an electrical connector that is connected to an external power source such as household electric current. A two hour timer neutral line 147 provides a neutral line to the two hour timer. A four hour timer neutral line 141 provides a neutral line to the four hour timer. A six hour timer neutral line 140 provides a neutral line to the six hour timer.

A first ozone generator timer neutral line 143 provides a neutral line for a first ozone generator, and a second ozone generator timer neutral line 144 provides a neutral line for a second ozone generator. An ozone generator fan neutral line 142 provides a neutral line for an ozone generator fan. A vapor timer neutral line 145 provides a neutral line for a vapor timer. A door latch control line 146 provides a neutral line for a door latch. A ground fault interrupt 15 amp neutral line 148 provides a neutral line for a ground fault interrupt of 15 A. And exhaust timer neutral line 150 provides a neutral line for an exhaust timer. An exhaust fan neutral line 151 provides a neutral line for an exhaust fan. An exhaust on light neutral line 152 provides a neutral line for an exhaust on light. A vapor unit neutral line 153 provides a neutral line for a vapor unit. A vapor on light neutral line 154 provides a neutral line for a vapor on light. An ozone strobe light neutral line 155 provides a neutral line for an ozone strobe light. An ozone generator on light neutral line 156 provides a neutral line for an ozone generator. A power on light neutral line 157 provides a neutral line for a power on light. A low-voltage 12 V transformer neutral line 158 provides a neutral line for a low-voltage 12 V

1. A decontamination appliance comprising:
a control cabinet having a control circuit;
a decontamination cabinet;
an ozone generator, wherein the ozone generator supplies ozone to the decontamination cabinet, wherein the ozone generator is controlled by the control circuit;
a hydrogen peroxide vaporizer, wherein the hydrogen peroxide vaporizer is also controlled by the control circuit; and
a circulation fan, wherein the circulation fan circulates a stream of air through the decontamination cabinet, wherein the circulation fan circulates ozone generated from the ozone generator and hydrogen peroxide paper generated from the hydrogen peroxide vaporizer.

2. The decontamination appliance of claim 1, wherein the decontamination cabinet further includes trays mounted on a tray rack.

3. The decontamination appliance of claim 1, wherein the decontamination appliance further includes a humidifier.

4. The decontamination appliance of claim 1, wherein the decontamination cabinet further includes a door lock, wherein the door lock is controlled by the control circuit.

5. The decontamination appliance of claim 1, wherein the decontamination cabinet further includes a catalytic converter chamber, wherein the door lock is controlled by the control circuit.

6. The decontamination appliance of claim 1, wherein the decontamination cabinet further includes a catalytic converter chamber, wherein the control circuit further includes a three position switch that selects between different modes of operation including a first mode having a first mode duration, a second mode having a second mode duration that is longer than the first mode duration, and a third mode having a third mode duration that is longer than the second mode duration.

7. The decontamination appliance of claim 1, wherein the control circuit is configured to start a cycle by first activating the hydrogen peroxide vaporizer, and then activating the ozone generator, wherein the cycle is a preset program.
